(19) United States
(12) Patent Application Publication
(10) Pub. No.: US 2005/0112606 A1
(43) Pub. Date: May 26, 2005

(54) Advanced microfluidics

(76) Inventors: Martin Fuchs, Uxbridge, MA (US); Jonathan Larson, New Ipswich, NH (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/821,664

(22) Filed: Apr. 9, 2004

Related U.S. Application Data
(60) Provisional application No. 60/461,851, filed on Apr. 10, 2003.

(51) Int. Cl. C12Q 1/68; C12M 1/34

(52) U.S. Cl. 435/6; 435/287.2

(57) ABSTRACT

A microfluidic device for altering the flow of a carrier fluid containing a polymer, such that, the polymer can be positioned, aligned, or elongated. The device accomplishes these effects by directing the carrier fluid in a laminar flow into obstacles, or other fluids to knowingly alter the path of the carrier fluid streamlines. These streamlines, in turn, apply fluidic drag forces against the polymer to manipulate it into a desired configuration. Other aspects of the device retain a polymer in an aligned or elongated state with crimps which prevent portions of the polymer from coiling. These structures utilize the natural concept of increasing entropy to allow small portions of an aligned or elongated polymer to return to a high entropy, or coiled state, while retaining the majority of the polymer in a low entropy, aligned or elongated state for subsequent analysis or manipulation.

### RELATED APPLICATIONS

This application claims the benefit of U.S. provisional patent application Ser. No. 60/461,851, filed Apr. 10, 2003.

### FIELD OF THE INVENTION

The present invention relates to a polymer manipulation device and more particularly to a device for positioning a polymer, aligning a polymer, elongating a polymer or retaining a polymer in an elongated or aligned state.

### BACKGROUND OF THE INVENTION

The study of molecular and cellular biology is focused on the microscopic structure of cells. It is known that cells have a complex microstructure that determines the functionality of the cell. Much of the diversity associated with cellular structure and function is due to the ability of a cell to assemble various building blocks into diverse chemical compounds. The cell accomplishes this task by assembling polymers from a limited set of building blocks referred to as monomers. One key to the diverse functionality of polymers is based in the primary sequence of the monomers within the polymer. This sequence is integral to understanding the basis for cellular function, such as why a cell differentiates in a particular manner or how a cell will respond to treatment with a particular drug.

The ability to identify the structure of polymers by identifying their sequence of monomers is integral to the understanding of each active component and the role that component plays within a cell. By determining the sequences of polymers it is possible to generate expression maps, to determine what proteins are expressed, to understand where mutations occur in a disease state, and to determine whether a polysaccharide has better function or loses function when a particular monomer is absent or mutated.

### SUMMARY OF THE INVENTION

The microfluidic devices of the present invention are adapted to orient and/or manipulate a polymer or group of polymers in a various manners. These may include positioning, aligning, elongating one or more polymers, or retaining one or more polymers in an aligned or elongated state. It is sometimes useful to manipulate a polymer in such a manner so that its structure can be identified more easily in a subsequent analysis, or so that its structure can by analyzed while it is being manipulated. Thus, the devices and methods of the invention are useful for analyzing polymers.

In one embodiment, an apparatus for positioning a polymer in a microchannel is disclosed. The apparatus includes a microchannel with first and second ends and substantially opposed sidewalls. The microchannel is constructed and arranged to transport a polymer carrier fluid such that, when present, the polymer flows from the first end toward the second end in a laminar flow stream. The apparatus has a first section of the microchannel disposed between the first and second ends of the microchannel. The substantially opposed sidewalls of the first section are constructed and arranged to create a first velocity gradient in the flow stream passing there through. Opposed flow control channels are in fluid communication with the microchannel and the flow channels are positioned between the first section and the second end of the microchannel. A flow controller controls the flow of fluid through the opposed flow control channels to maintain the flow stream containing the polymer in a laminar state isolated from the substantially opposed sidewalls of the microchannel at points downstream from the opposed flow control channels. The apparatus also has a second section of the microchannel disposed between the opposed flow control channels and the second end of the microchannel. The substantially opposed sidewalls of the second section are constructed and arranged to create a second velocity gradient in the flow stream passing there through. A detection zone is also disposed within the microchannel.

Also disclosed is a method of positioning a polymer within a microchannel. The method comprises providing a polymer positioning apparatus including a microchannel with first and second ends and substantially opposed sidewalls. The microchannel is constructed and arranged to transport a polymer carrier fluid such that, when present, the polymer flows from the first end toward the second end in a laminar flow stream. The apparatus has a first section of the microchannel disposed between the first and second ends of the microchannel. The substantially opposed sidewalls of the first section are constructed and arranged to create a first velocity gradient in the flow stream passing there through. Opposed flow control channels are in fluid communication with the microchannel and the flow channels are positioned between the first section and the second end of the microchannel. A flow controller controls the flow of fluid through the opposed flow control channels to maintain the flow stream containing the polymer in a laminar state isolated from the substantially opposed sidewalls of the microchannel at points downstream from the opposed flow control channels. The apparatus also has a second section of the microchannel disposed between the opposed flow control channels and the second end of the microchannel. The substantially opposed sidewalls of the second section are constructed and arranged to create a second velocity gradient in the flow stream passing there through. A detection zone is also disposed within the microchannel. The method also includes providing a polymer carrier fluid containing a polymer into the microchannel and manipulating the flow controller for selectively positioning the polymer within the microchannel.

In another embodiment, a method for elongating a polymer is disclosed. The method comprises providing a carrier fluid containing a polymer to a microchannel adapted to deliver a polymer from a first end of the microchannel to a second end of a microchannel. Focusing the carrier fluid in a first velocity gradient created by a first set of substantially opposed walls of the microchannel. Focusing the carrier fluid in a second velocity gradient created by a side flow of fluid entering the microchannel and then focusing the carrier fluid in a third velocity gradient created by a second set of substantially opposed walls of the microchannel.

In an additional embodiment, an apparatus for elongating a polymer is disclosed which comprises a microchannel having a first and second end, a polymer elongation zone, and opposed sidewalls. The microchannel is constructed and arranged to transport a polymer carrier fluid such that, when present, the polymer flows from the first end toward the polymer elongation zone in a laminar flow stream. Opposed flow control channels are in fluid communication with the microchannel through the opposed sidewalls. The flow control channels are positioned between the first end of the microchannel and the polymer elongation zone. Opposed polymer control channels are in fluid communication with the microchannel through the opposed sidewalls and define the polymer elongation zone. They are positioned between the opposed flow control channels and the second end of the microchannel. The apparatus has a first end fluid controller for directing a fluid through the microchannel from the first end toward the polymer elongation zone, an opposed flow controller for controlling the flow of fluid through the opposed flow control channels to maintain the flow stream containing the polymer in a laminar state isolated from the opposed sidewalls of the microchannel, an opposed polymer channel controller for controlling the flow of fluid through the opposed polymer control channels, and a second end flow controller for directing fluid through the microchannel from the second end toward the polymer elongation zone.

Also described is a method for elongating a polymer which comprises providing a polymer elongation apparatus having a microchannel with a first end, a polymer elongation zone, and opposed sidewalls. The microchannel is constructed and arranged to transport a polymer carrier fluid such that, when present, the polymer flows from the first end toward the polymer elongation zone in a laminar flow stream. The apparatus also has opposed flow control channels in fluid communication with the microchannel through the opposed sidewalls. The flow control channels are positioned between the first end of the microchannel and the polymer elongation zone. Opposed polymer control channels are in fluid communication with the microchannel through the opposed sidewalls. The polymer control channels define the polymer elongation zone and are positioned between the opposed flow control channels and the second end of the microchannel. The apparatus also utilizes an opposed flow controller for controlling the flow of fluid through the opposed flow control channels to maintain the flow stream containing the polymer in a laminar state isolated from the opposed sidewalls of the microchannel. The apparatus also uses an opposed polymer channel controller for controlling the flow of fluid through the opposed polymer control channels. The method also includes directing a fluid carrier containing the polymer to be elongated through the microchannel from the first end toward the polymer elongation zone in a laminar flow stream. A flow control fluid is directed through the opposed flow control channels into the microchannel in a manner such that polymer-containing flow stream is isolated from the sidewalls of the microchannel.

In another aspect, an apparatus is disclosed for maintaining a polymer in an elongated configuration. The apparatus comprises a microchannel constructed and arranged to contain a polymer carrier fluid. The microchannel has opposed sidewalls defining a first microchannel width, a second microchannel width, smaller than the first width, and a transition between the first and second microchannel widths. The transition is adapted to contact and inhibit relaxation of an elongated polymer contained within the first microchannel width.

Yet another embodiment is an apparatus for elongating a polymer and maintaining it in an elongated configuration. The apparatus comprises a microchannel having first and second ends, a polymer elongation zone, and opposing sidewalls. The microchannel is also constructed and arranged to transport a polymer carrier fluid such that, when present, the polymer flows from the first end toward the polymer elongation zone in a laminar flow stream. Opposed polymer control channels are in fluid communication with the microchannel through the opposing sidewalls. The polymer control channels are adapted to provide a flow of fluid for defining the polymer elongation zone. The polymer control channels are positioned between the first end and the second end of the microchannel, wherein at least one of the polymer control channels includes at least one transition to a narrower microchannel width. The transition is for contacting and inhibiting relaxation of an elongated or aligned polymer contained in the narrower width. Furthermore, at least one of the polymer control channels also includes at least one serpentine bend to cause at least one portion of the polymer control channel to be located adjacent and parallel to another portion of the polymer control channel. The apparatus also comprises a first end fluid controller for directing a fluid through the microchannel from the first end toward the polymer elongation zone.

In one embodiment, an apparatus for detecting a polymer is disclosed. The apparatus includes a microchannel having first and second ends. The apparatus also includes an obstacle field arranged between the first and second ends at the microchannel. The microchannel is constructed and arranged to transport a polymer carrier fluid such that, when present, the polymer flows from the first end, through the obstacle field and toward the second end in a laminar flow, and a detection zone located in the obstacle field, the detection zone for detecting the polymer. Also disclosed is a method for detecting a polymer, by applying a polymer to the above mentioned apparatus and then detecting the polymer.

Another disclosed embodiment is directed to a method for detecting a polymer. The method comprises providing an apparatus comprising a microchannel having first and second ends and an obstacle field between the first and second ends. The microchannel is constructed and arranged to transport the polymer carrier fluid such that, when present, the polymer flows from the first end, through the obstacle field and toward the second end in a laminar flow. The method includes providing a polymer carrier fluid containing a polymer to be detected, and then flowing the polymer carrier through the obstacle field in a manner such that at least one polymer becomes transiently tethered to at least one obstacle comprising the obstacle field and then detecting the transiently tethered polymer.

In one additional embodiment, an apparatus for holding a polymer on a microchip is disclosed. The apparatus comprises a microchannel disposed on the microchip, where the microchannel has a first end and a second end and opposing sidewalls. The microchannel is constructed and arranged to transport a polymer in a carrier fluid, such that, when present, the polymer flows from the first end toward the second end along a flow path. The microchannel is also arranged on the microchip with at least one bend to cause a first portion of the microchannel to be located adjacent to and aligned with a second portion of the polymer control channel.

Further features and advantages of the present invention, as well as the structure of various embodiments, are described in detail below with reference to the accompanying drawings.

### BRIEF DESCRIPTION OF THE DRAWINGS

Various embodiments of the invention will now be described, by way of example, with reference to the accompanying drawings, in which:

FIG. 1 is a view of a polymer in a coiled configuration;

FIG. 2 is a view of a polymer in a hairpinned configuration;

FIG. 3 is a view of a polymer in an elongated configuration;

FIG. 4 is a graph of applied elongation force versus percent of unstretched contour length for typical polymers;

FIG. 5 is a view of laminar fluid flow with descriptive streamlines shown therein;

FIG. 6 is a view of laminar flow turning turbulent after contacting an object;

FIG. 7 is a view of uniform velocity laminar fluid moving a coiled polymer disposed therein;

FIG. 8 is a view of laminar fluid flowing about an object placed therein;

FIG. 9 is a view of laminar fluid flowing about a polymer anchored at one end;

FIG. 10 is a view of two streamlines depicting a fluid in sheer and a polymer in the sheer zone;

FIG. 11 is another view of two streamlines depicting a fluid in sheer and a polymer in the sheer zone;

FIG. 12 is a view of fluid streamlines being focused in a velocity gradient;

FIG. 13 is a view of laminar fluid impinging an object and creating a stagnation point;

FIG. 14 is a view of two opposed laminar flows impinging one another;

FIG. 15 is a top view of a microchannel having opposed flow control channels according to one embodiment of the invention;

FIG. 16 is a top view of a microchannel having opposed flow control channels according to another embodiment of the invention;

FIG. 17 is a top view of a microchannel having opposed polymer control channels;

FIG. 18 is a top view of a microchannel having opposed flow control channels and opposed polymer control channels;

FIG. 19 is a top view of a microchannel having opposed flow control channels and opposed polymer control channels according to another aspect of the invention;

FIG. 20 is a top view of a microchannel having two different widths for inhibiting the relaxation of an elongated or aligned polymer;

FIG. 21 is a top view of a microchannel having multiple sections of different widths for inhibiting the relaxation of an elongated or aligned polymer;

FIG. 22 is a top view of a microchannel having a serpentine section and different dimensions for inhibiting the relaxation of an elongated polymer;

FIG. 23 is a side view of a microchannel having two different dimensions for inhibiting the relaxation of an elongated polymer;

FIG. 24 is a top view of a microchannel having opposed flow control channels, opposed polymer control channels, and two different dimensions for inhibiting the relaxation of an elongated polymer; and

FIG. 25 is a top view of a microchannel having a first section for creating a velocity gradient, opposed flow control channels, and a second section for creating a second velocity gradient.

### DETAILED DESCRIPTION

The microfluidic device of the present invention is adapted to deliver a fluid containing a polymer through a microchannel such that, when present, the polymer can be positioned, aligned, elongated, or inhibited from relaxing from an aligned or elongated state. Such functions performed on the polymer are useful in preparing the polymer for analysis.

The term “analyzing a polymer” as used herein to means obtaining some information about the structure of the polymer such as its size, the order of its units, its relatedness to other polymers, the identity of its units, or its presence or absence in a sample. Since the structure and function of biological polymers are interdependent, the structure can reveal important information about the function of the polymer.

A “polymer” as used herein is a compound having a linear backbone of individual units which are linked together by linkages. In some cases, the backbone of the polymer may be branched. Preferably the backbone is unbranched. The term “backbone” is given its usual meaning in the field of polymer chemistry. The polymers may be heterogeneous in backbone composition thereby containing any possible combination of polymer units linked together such as peptide- nucleic acids (which have amino acids linked to nucleic acids and have enhanced stability). In one embodiment the polymers are, for example, polynucleic acids, polypeptides, polysaccharides, carbohydrates, polyurethanes, polycarbonates, polyureas, polyethyleneimines, polyarylene sulfides, polysiloxanes, polyimides, polyacetates, polyamides, polyesters, or polythioesters. In the most preferred embodiments, the polymer is a nucleic acid or a polypeptide. A “nucleic acid” as used herein is a biopolymer comprised of nucleotides, such as deoxyribose nucleic acid (DNA) or ribose nucleic acid (RNA). A polypeptide as used herein is a biopolymer comprised of linked amino acids.

As used herein with respect to linked units of a polymer, “linked” or “linkage” means two entities are bound to one another by any physicochemical means. Any linkage known to those of ordinary skill in the art, covalent or non-covalent, is embraced. Natural linkages, which are those ordinarily found in nature connecting the individual units of a particular polymer, are most common. Natural linkages include, for instance, amide, ester and thioester linkages. The individual units of a polymer analyzed by the methods of the invention may be linked, however, by synthetic or modified linkages. Polymers where the units are linked by covalent bonds will be most common but may also include hydrogen bonded units, etc.

The polymer is made up of a plurality of individual units. An “individual unit” as used herein is a building block or monomer which can be linked directly or indirectly to other building blocks or monomers to form a polymer. The polymer preferably is a polymer of at least two different linked units. The at least two different linked units may produce or be labeled to produce different signals.

The “label” may be, for example, light emitting, energy accepting, fluorescent, radioactive, or quenching as the invention is not limited in this respect. Many naturally occurring units of a polymer are light emitting compounds or quenchers, and thus are intrinsically labeled. The types of labels are useful according to the methods of the invention. Guidelines for selecting the appropriate labels, and methods for adding extrinsic labels to polymers are provided in more detail in U.S. Pat. No. 6,355,420 B1.

The signal detection methods may include methods such as nanochannel analysis (US Genomics, Woburn, Mass.), near-field scanning microscopy, atomic force microscopy, scanning electron microscopy, waveguide structures, or other known methods as the invention is not limited in this respect.

Once the signal is generated it can then be detected and analyzed. The particular type of detection means will depend on the type of signal generated, which of course will depend on the type of interaction that occurs between a unit specific marker and an agent. Many interactions involved in methods of the invention will produce an electromagnetic radiation signal. Many methods are known in the art for detecting electromagnetic radiation signals, including two- and three-dimensional imaging systems.

Optical detectable signals are generated, detected and stored in a database. The signals can be analyzed to determine structural information about the polymer. The signals can be analyzed by assessing the intensity of the signal to determine structural information about the polymer. The computer may be the same computer used to collect data about the polymers, or may be a separate computer dedicated to data analysis. A suitable computer system to implement embodiments of the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection mechanism. Computer programs for data analysis of the detected signals are readily available from CCD (Charge Coupled Device) manufacturers.

Other interactions involved in methods of the invention will produce a nuclear radiation signal. As a radiolabel on a polymer passes through the defined region of detection, nuclear radiation is emitted, some of which will pass through the defined region of radiation detection. A detector of nuclear radiation is placed in proximity of the defined region of radiation detection to capture emitted radiation signals. Many methods of measuring nuclear radiation are known in the art including cloud and bubble chamber devices, constant current ion chambers, pulse counters, gas counters (i.e., Geiger-Müller counters), solid state detectors (surface barrier detectors, lithium-drifted detectors, intrinsic germanium detectors), scintillation counters, Cerenkov detectors, to name a few.

Other types of signals generated are well known in the art and have many detections means which are known to those of skill in the art. Some of these include opposing electrodes, magnetic resonance, and piezoelectric scanning tips. Opposing nanoelectrodes can function by measurement of capacitance changes. Two opposing electrodes create an area of energy storage, located effectively between the two electrodes. It is known that the capacitance of such a device changes when different materials are placed between the electrodes. This dielectric constant is a value associated with the amount of energy a particular material can store (i.e., its capacitance). Changes in the dielectric constant can be measured as a change in the voltage across the two electrodes. In the present example, different nucleotide bases or unit specific markers of a polymer may give rise to different dielectric constants. The capacitance changes as the dielectric constant of the unit specific marker of the polymer per the equation: C=KCₒ, where K is the dielectric constant and Cₒ is the capacitance in the absence of any bases. The voltage deflection of the nanoelectrodes is then outputted to a measuring device, recording changes in the signal with time.

An embodiment of the microfluidic device has flow control channels that provide flow through opposed sidewalls of a microchannel. Such opposed flow can alter the flow of fluid containing the polymer is the microchannel to either position the polymer, align the polymer or to elongate the polymer.

Other embodiments of the microfluidic device have polymer control channels that separate streamlines in the flow of the fluid containing the polymer. A polymer with portions located in the separated streamlines can be aligned or elongated as the portions are separated from one another. The separated streamlines can also be used to direct a polymer contained therein in a direction associated with either of the separated streamlines.

Still, other embodiments of the microfluidic device have a microchannel with an obstacle field disposed therein. The obstacle field can serve to separate streamlines of a carrier fluid that impinge the obstacles in the obstacle field. The separated streamlines, in turn, serve to align or partially align any polymers that have contacted the obstacles. A detection zone can also be placed in the obstacle field for detecting the polymers as they contact and move about the obstacles.

Embodiments of the microchannel of the microfluidic device can also have cross sections of different dimensions for retaining a polymer in a substantially aligned or elongated state. This can occur by having portions of a polymer disposed within a microchannel cross section of a smaller dimension, thereby inhibiting the relaxation of elongated or aligned polymers. Microchannels can have multiple cross sections of different dimensions so they can accommodate polymers of various lengths. The microchannel may also be arranged in a serpentine fashion to hold a long polymer in an organized coil. The polymer may be analyzed when it is retained in an elongated or aligned state or it may be held for additional preparatory steps to be performed before or between analysis steps.

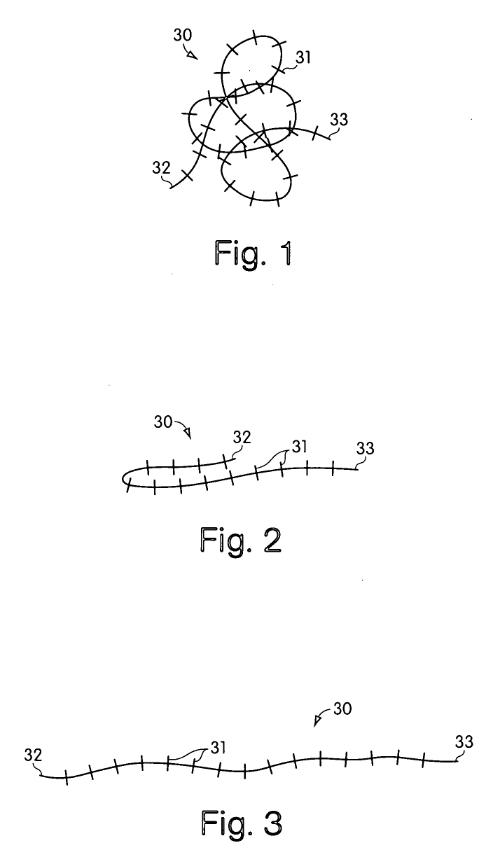

Turning now to the Figures, and in particular FIGS. 1-3 where a polymer 30 is illustrated in three different states of interest. FIG. 1 shows a polymer in a coiled or “balled-up”, high entropy state. FIG. 2 shows a polymer in a hairpinned, low entropy state. FIG. 3 shows the polymer in an aligned low entropy state. Entropy is very generally the measure of disorder in a system, the system in this case being a polymer. In this manner, entropy is indicative of how coiled or tangled a polymer is with itself. For a polymer to be arranged in a low entropy state as shown in FIG. 3, forces need to be applied to the polymer to force the molecule into a more ordered state. For instance, a polymer subjected to elongational flow or other forces that cause linearization begins to deform and form a highly ordered state when the force exceeds the entropic elasticity that tends to coil it. Such a high degree of order is unlikely to occur naturally, because specific forces must be applied to affect the inter- and intra-molecular interactions involved in the tertiary structure of the molecule.

The entropy of systems normally increases over time unless the system is otherwise acted upon to maintain or create a lower entropy state. If a polymer is caused to form such an ordered state, the natural tendency for entropy to increase within a system will eventually result in the polymer returning to a coiled state.

It is now possible to detect and analyze a polymer when the polymer is in an aligned or elongated state similar to that shown in FIG. 3. U.S. Pat. No. 6,355,420, which is hereby incorporated by reference, describes methods for linear analysis of polymers. The methods described therein provide methods for rapid detection of different components that comprise the polymer.

“Contour length”, as discussed herein is a parameter used to characterize a polymer. The contour length of a polymer is its length measured from a first end 32 to a second end 33 of the polymer 30 by tracing the polymer unit to unit while the polymer is in an unstretched state. The “apparent length” of a polymer as used herein is the shortest distance between the first end 32 and the second end 33. Apparent length is measured along a direct line between the first end 32 and the second end 33 of a polymer, meaning that it can be significantly shorter than contour length when a polymer is coiled or hairpinned. When a polymer is aligned yet not elongated, its apparent length will be substantially the same as its contour length. Most DNA and RNA have individual units or base pairs that are approximately 3.4 Å in length. For these polymers, contour length can be calculated by multiplying the number of base pairs by 3.4 Å.

The term “aligned” as used herein is used to describe a polymer with its units arranged in a substantially linear fashion. The term “elongated” as used herein is generically used to describe a polymer, or portion of a polymer that exists at greater than substantially 90% of its contoured length. An elongated polymer or portion of a polymer is necessarily also aligned. The terms “partially stretched”, “stretched”, and “over stretched” refer to specific degrees of alignment or elongation as is discussed below.

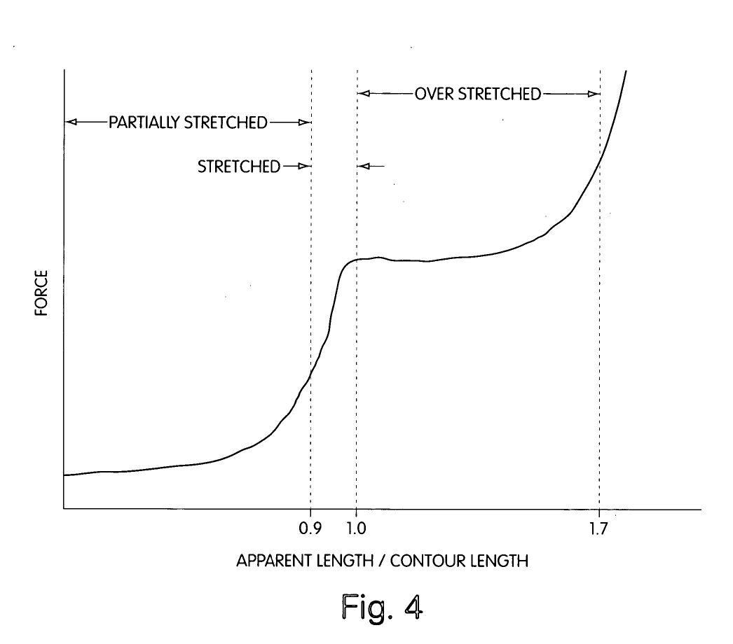

Many polymers, such as DNA can be elongated beyond their contour length. FIG. 4 depicts the force associated with elongating a double strand of DNA from its native “balled-up” or coiled state to an aligned state of full contour length and then beyond to the shape of S-DNA. The X-axis of FIG. 4 represents the ratio of apparent length over contour length of a double strand of DNA. The Y-axis represents the magnitude of an elongational force applied to the double strand of DNA. Dimensions are not included on the Y-axis, however, points further from the X-axis represent a force of greater magnitude. The relatively flat (horizontal) points on the curve near the Y-axis represent DNA in its “balled-up”, or in its coiled, native state. The DNA has base pair spacing of approximately 3.4 Å in this state. Points along the curve that are further from the Y-axis yet still on the substantially horizontal portion of the curve represent DNA and up to a ratio of about 90%, that is partially untangled. In this state, the DNA (or RNA) still has base pair lengths of approximately 3.4 Å and is technically known as being “partially stretched”. As additional force is applied to the DNA (or RNA) it is formed into a linear configuration with an overall end-to-end length approximating its contour length. In this state, the DNA (or RNA) is characterized as being “stretched”. As additional forces are applied to the DNA (or RNA), it may become “over stretched”, with its base pairs being extended to lengths greater than approximately 3.4 Å each. As the graph depicts, over stretching does not initially incur much additional force to be applied to the DNA. However, after the DNA has been stretched to approximately 1.7 times its contour length, the force required to extend it any further increases dramatically. While FIG. 4 depicts a force versus elongation curve for DNA and not RNA, the terms “partially stretched”, “stretched”, and “over stretched” apply to both DNA and RNA.

FIG. 4 shows that it takes only nominal amounts of force to move DNA from a low apparent length to contour length ratio (high entropy) towards a higher apparent length to contour length ratio (lower entropy). This assumes that the DNA is elongated evenly over its entire length and that no portions of the polymer are partially stretched or over stretched.

However, when the apparent length of the DNA nears its contour length, the force required to elongate the polymer increases sharply. This steep portion of the curve may be used advantageously by embodiments of the present invention. In this manner, a force associated with a point along the steep portion of the curve near a ratio value of unity can be applied to a polymer, such as DNA, to stretch it without over stretching.

Once the polymer stretched, that is having its apparent length is substantially equivalent to its contour length, only a marginal amount of additional force is required to begin over stretching. This is represented by the substantially flat portion of the curve associated with ratio values greater than unity but less than 1.7. This portion of the curve represents untwisting of the strands of DNA. This untwisted state of DNA is sometimes referred to as S-DNA.

The curve again reaches a steep portion near ratio values of 1.7 where the individual units of the double-strand DNA are over stretched further apart from one another. Forces required to over stretch the polymer beyond this point continue to rise until they are great enough to break the polymer. The effects of solution conditions and stretching on DNA to produce over stretched S-DNA and beyond are described in the literature, for instance, Rouzina and Bloomfield, Biophysical Journal, 80:894 (2001), which is hereby incorporated by reference.

The “persistence length” of a polymer as described herein is a parameter that indicates the degree to which a polymer can become tightly coiled. The persistence length of a polymer is generally the length of the polymer over which the polymer will naturally remain aligned. A smaller persistence length means that a polymer is capable of being arranged in tighter turns. This coupled with the concept of entropy means that polymers with shorter persistence lengths will likely be found naturally in smaller coils of tighter turns. Generally, the persistence length is orders of magnitude smaller than the contour length for the polymers of concern. This once again suggests that the polymers will naturally reside in a highly coiled state.

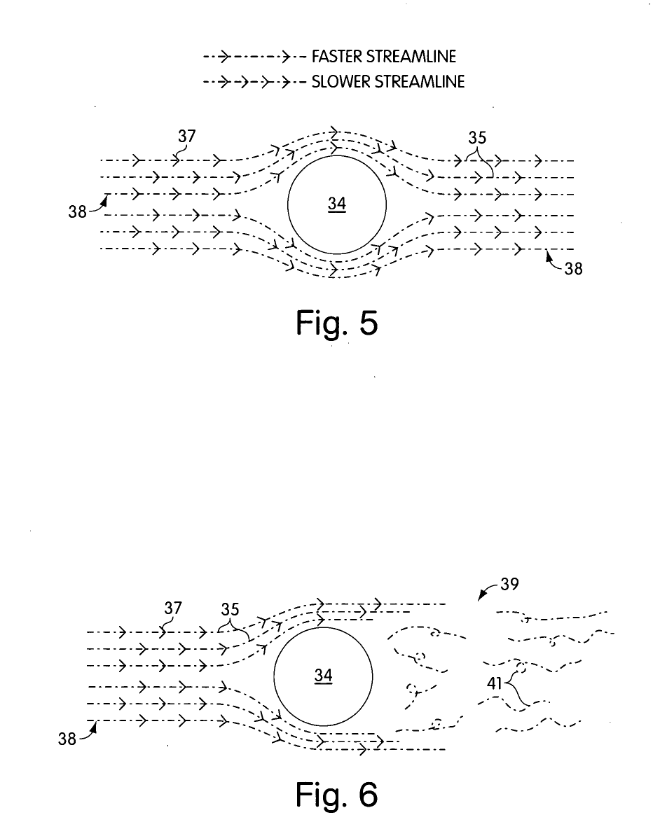

Various fluid terms are now described in a manner that relates to the microfluidic devices of the invention. “laminar flow” as used herein describes a flow in which the fluid moves in layers without fluctuations or turbulence so that successive particles passing the same point have a similar velocity. As shown in FIG. 5, laminar flow 38 is characterized by smooth streamlines 35 throughout the flow field. A streamline is a visualization of a line following the tangent to the velocity vector in a fluid field at an instant in time. Flow follows streamlines and cannot cross a streamline. FIG. 5 shows laminar flow past an object/obstacle 34 immersed in a fluid. The streamlines in this figure include arrows 37 which indicate the direction of the flow for a given streamline and also the velocity of the flow. The direction of the flow follows the arrow and the magnitude of the flows velocity is inversely proportional to the number of arrows per a given length, that is, fewer arrows per a given length means that a streamline is flowing faster. A streamline is shown to be accelerating as it moves downstream when it has arrows spaced further apart to one another at points downstream. The same convention associated with the streamlines of FIG. 5 is used throughout the figures in this application unless otherwise noted.

Streaklines are another visualization that can be used to describe the flow of a fluid. A streakline in a fluid represents the path that a given particle follows over time. For steady, laminar flow, the streaklines and streamlines will be coincident. However, laminar flow can have streaklines that differ from the streamlines if its streamlines are changing over time. Such flow is characterized as unsteady, laminar flow. In this regard, the streamlines 37 shown in the figures may also represent streaklines if the depicted flow is considered steady.

Unlike laminar flow, turbulent flow as depicted in FIG. 6, is characterized by streamlines and streaklines that often follow unpredictable paths. Streamlines of turbulent flow 39 often form eddies or vortices 41 that curl about themselves and one another over time, delivering the fluid to points downstream in a stochastic manner. FIG. 6 depicts flow impinging on an object 34 immersed in the fluid with the flow becoming turbulent 39 at points downstream from the object. Discontinuous looping streaklines shown at positions downstream from the object are the eddies and vortices that typically characterize turbulent flow. While the turbulent flow progresses generally in a downstream fashion, the specific path of any given particle is primarily random and unpredictable.

Reynolds number is a dimensionless parameter that describes fluid flow and whether it is in a laminar, or turbulent state. The equation for Reynolds number is shown below.

$$Re = \frac{ρ VD}{μ}$$

Laminar flow occurs at high viscosities, low velocities, low densities or small dimensions, which are factors used to determine Reynolds number. Laminar flow may turn turbulent when velocities or densities increase, or when viscosities decrease. Other dimensional factors such as sharp bends in a flow channel or interaction with small features may also cause laminar flow to trip into turbulent flow. A polymer immersed in a turbulent fluid will likely be randomly moved about in an unpredictable path as it moves downstream, unlike a polymer immersed in a laminar fluid that can be moved in a predictable fashion.

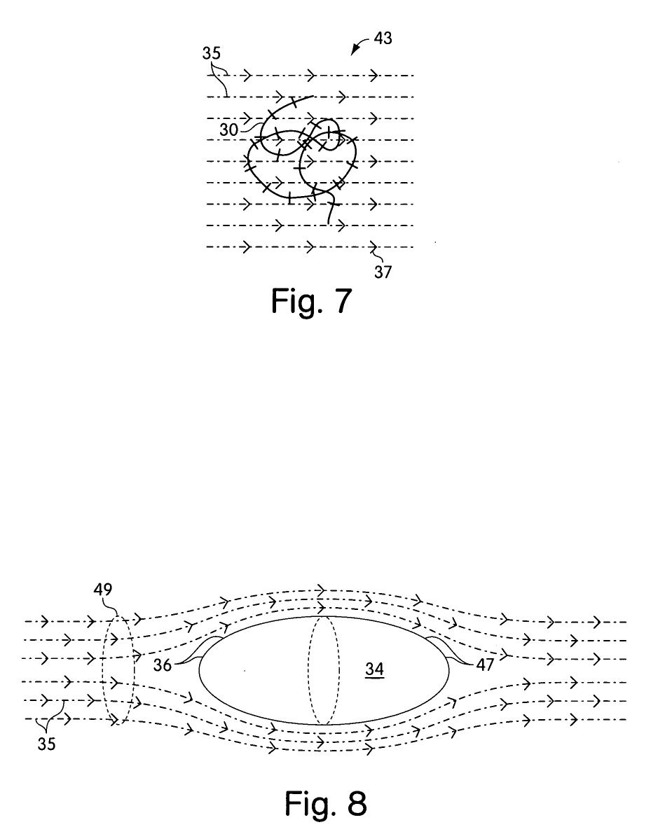

The term “uniform velocity laminar flow” as used herein describes the flow of a fluid without fluctuations such that successive particles passing the same point have a similar velocity and such that a particle will have the same velocity at points downstream. Uniform velocity laminar flow also means that adjacent streamlines will have similar velocities, as is illustrated in FIG. 7. Here a polymer is shown immersed in uniform velocity laminar flow 43 such that it can be moved along with the fluid without altering the orientation of the polymer. For instance, the polymer shown in FIG. 7 will remain in the position it is shown in as the uniform velocity laminar fluid carries the polymer 30 downstream. However, being located in a uniform velocity laminar fluid does not prevent a polymer from moving within the fluid. For instance, the same forces that might move a polymer in a still fluid, such as the forces associated with increasing entropy, can also move a polymer as it is travels in uniform velocity laminar flow.

The manner in which a fluid can manipulate a polymer contained therein is now discussed in general, and then for several specific scenarios. A polymer contained in a carrier fluid may be acted on by forces internal to the polymer, forces from any fluid in contact with the polymer, forces from any solid object contacting the polymer or by any body forces acting on the polymer, such as gravitational forces or buoyancy forces. The net effect of these forces determines where and how a polymer or a portion of the polymer move relative to the carrier fluid. In the absence of contact with another object, unbalanced internal forces, or body forces, a polymer contained in a uniform velocity laminar fluid will generally not move relative to the fluid. Each unit will instead follow the streamlines of the fluid until acted upon by another force as described above. In this manner, a polymer carried in such a uniform velocity laminar fluid moves relative to the fluid in a manner similar to the way it would move in a pool of still fluid. However, when the streamlines in contact with portions of the polymer move relative to one another or themselves, they apply forces to a portion of the polymer to move it into another position or configuration. It is this concept of altering streamlines to in turn alter the position or state of a polymer that is used by microfluidic devices of the present invention. Some of the ways in which a polymer can be affected by different streamlines, body forces, or contact forces will now be discussed.

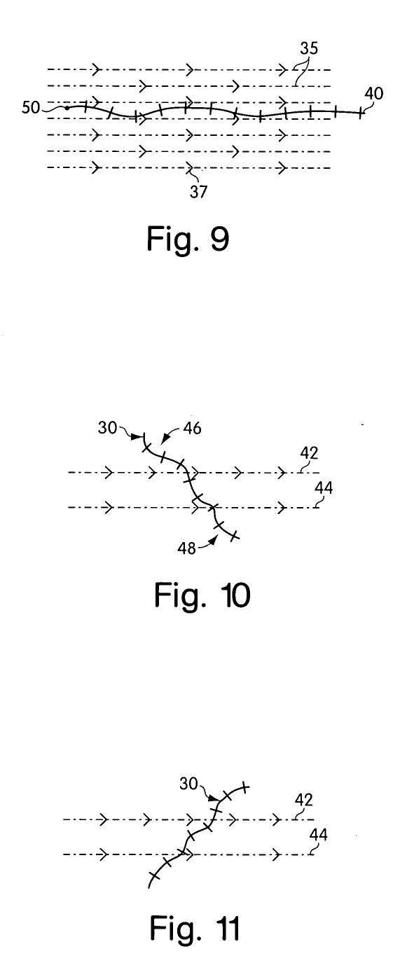

FIG. 8 shows an immersed object 34 and a fluid moving relative to it in a laminar flow. Both a pressure force and a fluidic drag force exist between the immersed object and the fluid. The pressure force is due to the difference between the higher pressure witnessed at the frontal contact area 36 between the object and the fluid and the lower pressure witnessed by the opposed trailing area 47. The magnitude of this force can generally be computed by integrating the difference is pressure over the projected cross-sectional area 49 of the object in a direction perpendicular to the direction of flow. Such a pressure force generally attempts to move the object with the fluid. For objects, such as polymers, with very high aspect ratios (where aspect ratio is the length of the polymer in the direction of flow divided by the diameter of the projected cross-sectional area), the pressure force is usually negligible when compared to the fluidic drag force. However, the pressure force can be great enough to push an anchored polymer, as shown in FIG. 9, towards an aligned state.

The fluidic drag force, as mentioned above, is the result of sliding contact between the object and the fluid. The fluidic drag force opposes the motion of the object within the fluid, that is, it attempts to move the object with the fluid. This force is also referred to as a fluidic friction force. The magnitude of a fluidic drag force is determined by several factors, most of which are also factors associated with the Reynolds number. One of such factors affecting the magnitude of drag force is the velocity of the fluid relative to the object, in this case a polymer. That is, a larger fluidic drag force will often be applied to a portion of a polymer in a fluid if the fluid velocity is increased with respect to the portion of the polymer. Other factors that determine the Reynolds number of a flow, and thus the fluidic drag force include the viscosity and density of a fluid and the contact area between the object and the fluid.

A fluidic drag force acts on an polymer in a distributed manner at all points where there is motion between the polymer and the fluid. A net fluid drag force is the sum of these forces integrated over the surface that the fluidic force is acting upon. The distributed fluidic drag forces can be used to align or elongate coiled polymers through the fluids that they are associated with. Aligning or elongating a polymer in this manner can be useful; however, the distributed nature of these forces can also create some challenges. For instance, consider a polymer immersed in a laminar flow and anchored at one end 50 as shown in FIG. 9. The fluid drag force will serve to align the polymer parallel to the streamlines of the fluid. This is accomplished when the fluidic drag force acts along the length of the polymer. In the scenario shown in FIG. 9, the net fluid drag force acting at any point of the polymer is the sum of the fluidic drag forces acting on all downstream points of the polymer. The graph of FIG. 9 also shows how this net fluidic drag force can increase along the length of the polymer for the case when one end 50 is anchored. In this scenario, the free end 40 of the polymer has relatively little net drag force acting upon it, which may not be enough force to stretch or even partially stretch the free end. As the net fluidic drag force increases along the polymer nearing the anchored end 50, it becomes adequate to align the polymer to a partially stretched or stretched state. The net force becomes much greater towards the anchored end 50, where it can be great enough to over stretch the polymer and potentially even break the polymer. This presents a challenge for polymers of significant length. First, if the velocity (or an equivalent parameter) is reduced to decrease the fluidic drag force, the free end of the polymer may not have enough net fluidic drag force applied to align it as desired. Second, portions of the polymer upstream from the free end will likely have high enough net fluidic drag forces to align them, but they may not be elongated as far as portions of the polymer that are further upstream. This situation can create a polymer that is not elongated consistently in places, having same portions coiled, particularly stretched, stretched, and/or over stretched. Third, the net fluidic force may be great enough to break the polymer at points distant from the free end 40 where the net force is too great.

A coiled polymer moving in a uniform velocity laminar fluid will remain in its coiled state absent any aligning forces acting upon it. However, when the streamlines of a fluid are moving relative to one another, a fluidic drag force will be applied to at least a portion of the polymer. One of such scenarios is shown in FIG. 10 where a slower streamline 42 is running adjacent to a faster streamline 44. Such streamlines are said to be in shear with one another. Here a polymer is shown with a first portion 46 located in the slower streamline and a second portion 48 located in the faster streamline. This polymer will experience a fluidic drag force from each of the streamlines as one or both of them and the corresponding portions of the polymer will be moving relative to one another. In the illustrated case, this force will serve to pull each portion of the polymer away from one another, which in this case aligns or elongates the polymer. FIG. 11 shows a scenario somewhat like that of FIG. 10 except that the streamlines and the polymer are arranged so the resulting fluidic drag forces serve to push the portions of the polymer toward one another, potentially coiling the polymer.

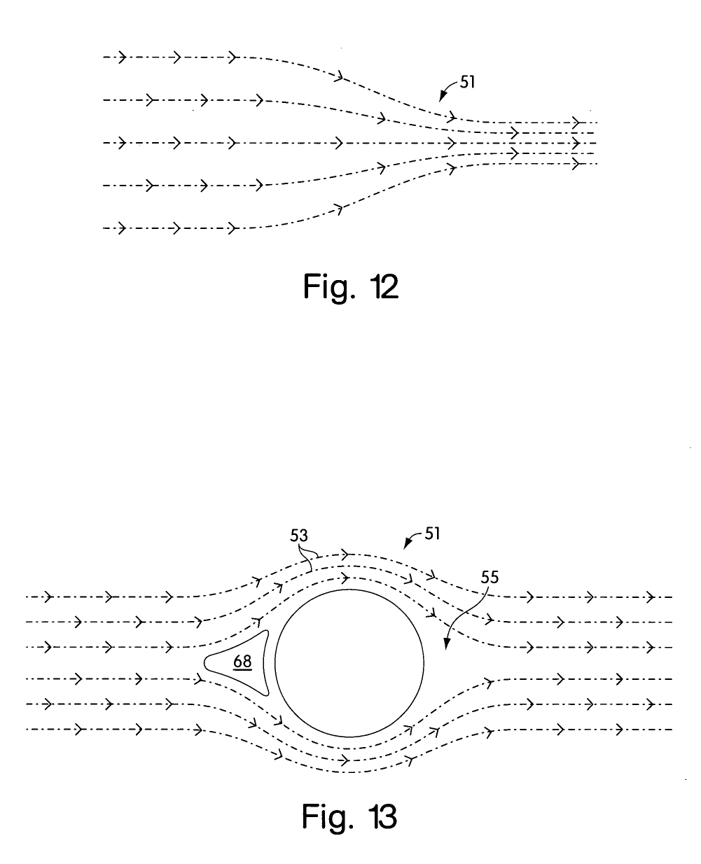

A velocity gradient as shown in FIG. 12, is another arrangement of laminar streamlines that can be used to manipulate a polymer. A velocity gradient 51 refers to streamlines or streaklines that reflect a fluid accelerating (or decelerating) as it passes from one point to another. A velocity gradient can occur in conjunction with some shear between adjacent streamlines, but does not have to. It is described herein without shear. It often occurs in conjunction with the streamlines being forced closer toward one another, or equivalently, being focused.

For incompressible fluids, which are fluids that occupy substantially the same volume when they are subjected to higher pressures, a velocity gradient is usually created by reducing the cross-sectional area of the flow path (in a direction perpendicular to the direction of flow) as shown in FIG. 12. The area reductions can be created by changes in the shape of a channel that contains a flowing fluid, such as by a funnel shape in a channel. They can also be created by introducing more fluid into an existing channel thereby reducing the cross-sectional area available for a given amount of fluid as it moves downstream. Reducing this area causes the fluid to accelerate to balance the volumetric flow rate at points upstream and downstream of the reduced cross-sectional area. FIG. 12 shows the acceleration of streamlines in a velocity gradient 51 as the streamlines are forced toward one another in a reduced cross-sectional area. Forcing these streamlines together causes them to accelerate. Any polymer contained in these streamlines as they are forced toward each other will likely be moved along with them, or equivalently, the polymer will be focused into a smaller cross sectional area perpendicular to the direction of flow. This effect can be useful in instances where a polymer needs to be targeted toward a specific location within a flow path.

A polymer entering a velocity gradient 51 can also be elongated in a direction parallel to the direction of flow. When a polymer enters into a velocity gradient, the forward-most portion of the polymer is pulled forward by the drag force of the accelerating fluid. The forward-most portion will continue to be pulled forward as long as it is located in the velocity gradient. Portions of the polymer that have not yet entered the velocity gradient may be pulled forward by the net fluidic drag force associated with the forward-most portion of the polymer as well as by the fluidic drag force acting on them as they enter the velocity gradient.

The effects of both focused streamlines and an associated velocity gradient are usually similar whether a polymer enters the gradient in a somewhat aligned state, a hairpinned state, a coiled state or any other configuration. Generally, the polymer will exit the gradient aligned or elongated in a direction parallel to flow and focused in a direction perpendicular to flow, yet still in a configuration similar to the way it entered the gradient. In this manner, focused streamlines can be used to focus a coiled polymer into a smaller cross-sectional area and a velocity gradient can be used to elongate its original configuration. It can elongate a polymer that enters the gradient in a somewhat aligned state, and even if the polymer is arranged in a hairpin fashion, sufficient duration in elongational flow may cause it to exit the gradient as an elongated, non-hairpinned polymer.

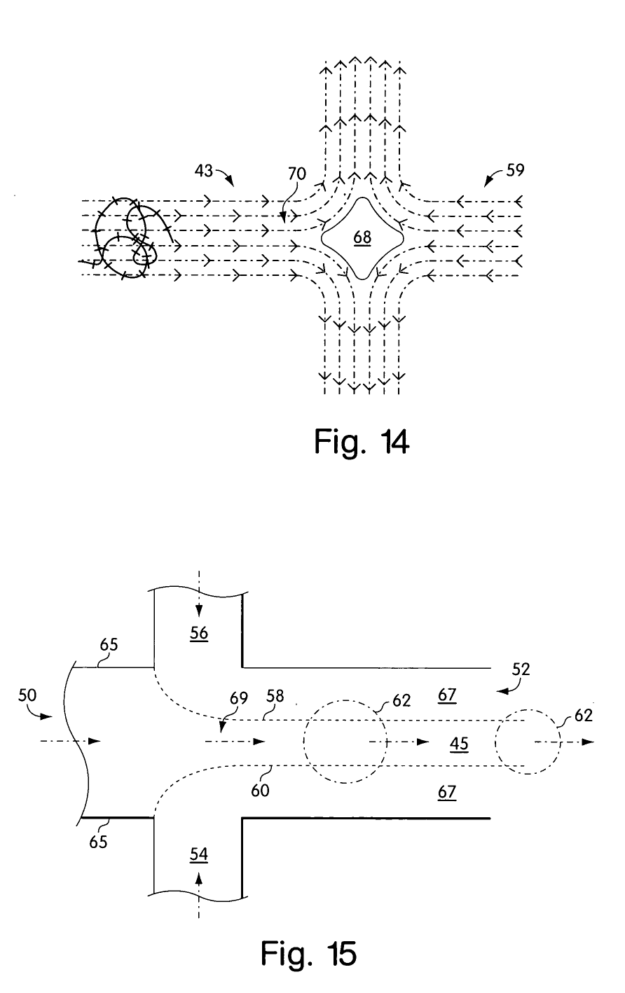

A stagnation point 68 is a fluidic occurrence that can be used to manipulate a polymer flowing in the fluid. When a fluid, particularly a laminar fluid impinges on an obstacle in its flow path, its streamlines 53 may separate and move around either side of the obstacle. The separated streamlines may continue around the obstacle and rejoin at a point directly downstream 55 from the obstacle as is shown in FIG. 13, or they may separate from the obstacle as they are flowing by, creating a turbulent zone 39 as is shown in FIG. 6. The streamlines may also be permanently separated if the obstacle does not allow them to come in contact again. The point where the streamlines contact the obstacle and separate about it is known as a stagnation point 68. It is termed this because the fluid exists at this point in low flow, or even no flow (stagnant) state. A stagnation point also occurs when a flow path impinges an obstacle like a wall. In this case, the streamlines will each follow a different course, presumably down separate channels after they pass the stagnation point. In another scenario, a stagnation point can be created by directing two flowing fluids against one another as shown in FIG. 14. Here, the streamlines of each fluid will meet with the streamlines of the opposing fluid 59, each separating at the stagnation point and in turn following different paths away from the stagnation point.

Stagnation points can be useful for aligning or elongating a polymer from a coiled state. For instance, consider a coiled polymer with portions located in laminar streamlines that separate upon nearing a stagnation point associated with an obstacle or an opposed flow stream. The separating streamlines will pull any portions of the polymer they contain with a fluidic drag force. The area adjacent the stagnation point where the streamlines separate is called an elongation zone 70. In cases where the coiled polymer enters the elongation zone with substantially equal portions of the polymer on either side of the stagnation point, as is shown in FIG. 14, the polymer may be elongated into an aligned or elongated, low entropy state by pulling the portions of the polymer away from one another.

Electrical devices may be used in combination with microfluidic devices of the present invention to accomplish various effects. For instance, electrical devices may be used to establish an electrical field across any portion of a microchannel, or an entire microchannel to help manipulate a polymer. Some polymers, such as DNA or RNA, may contain an electrical charge that allows them to be manipulated by an electrical field. Other polymers that may not naturally have an electrical charge can have a charge applied to them by any known manner. In one particular embodiment, such an electrical field may be useful in drawing portions of a polymer toward opposed sidewalls of the microchannel. This can assist a polymer in contacting an obstacle or a stagnation point 68 with substantially equal portions one either side of the obstacle or stagnation point. In other embodiments, an electrical field may be used to help maintain a polymer in aligned or elongated state.

A few of the various microfluidic devices used to create the above described fluidic phenomenon are now described. Most often these fluidic devices comprise microchannels that are manufactured through standard chip manufacturing technology. Most of these microchannels have a rectangular cross-section with a bottom wall 61 and opposed side walls 65 although other configurations are possible as the invention is not limited in this respect. The top wall 63 of these microchips is usually provided by a cover slip that can be fused over the base of the microchip or held in place by other means. The microchips provide a convenient medium for performing manipulation or analysis of polymers. Once the analysis is complete, the microchip can easily be discarded and replaced with a new one. However, some microchips may also be designed to be re-usable.

A microchip holder may be used to retain the microchip in a form that is easier to handle by the user. The holder may also be designed to mate with an analysis apparatus which accepts the holder and performs the analysis on the polymer. Such an analysis apparatus may provide the fluid that flows through the microfluid device and the polymers that are carried therein. This apparatus may be equipped with controls for manipulating the flow of fluid through the microchip and imaging equipment used to analyze the polymer once it is in its desired state the apparatus may also be used to monitor the polymer while it is being manipulated. This same apparatus may also include equipment to pre-process the polymers such that they may be analyzed. For instance, this apparatus may be capable of providing fluorescent dies, probes, etc. that are used in the analysis process. Such methods are known to those of skill in the art. For example, methods for analyzing linearized polymers, imaging devices, labeling methods, and strategies, etc. are described in U.S. Pat. No. 6,355,420 B1 which is hereby incorporated by reference.

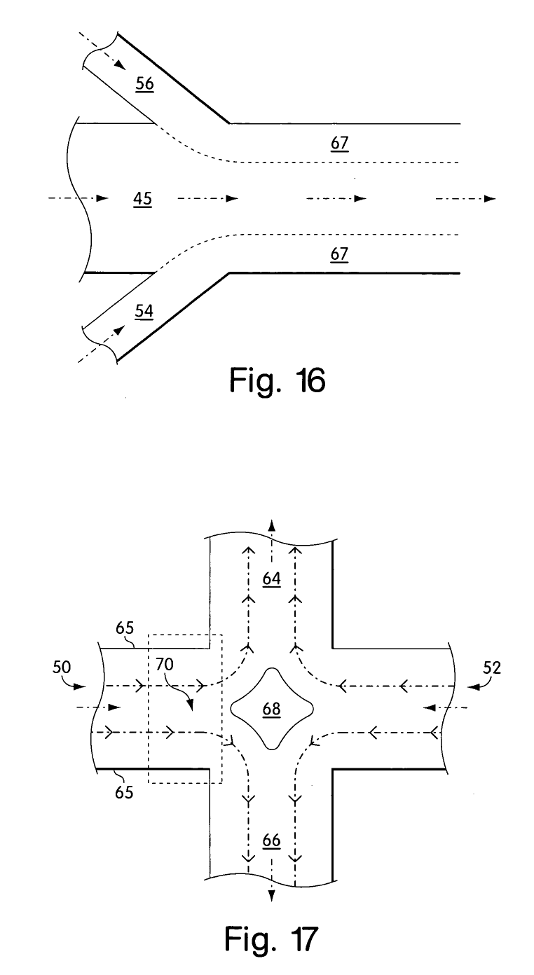

FIG. 15 shows one particular microfluidic device in the form of a microchannel formed in a microchip. The microchannel has a first end 50 and a second end 52 and is capable of delivering a carrier fluid that contains a polymer from the first end towards the second end. The microchannel is arranged to deliver the carrier fluid in a laminar state, although some turbulence may exist between the side walls, the bottom walls, the top wall or other edges in the microchannel without adversely affecting the performance of the device. Two opposed flow control channels 54, 56 connect to the microchannel through each of its opposed side walls 65. Each of these opposed flow control channels provide a side flow of fluid that enters the microchannel where the carrier fluid resides. The upper 58 and lower boundaries 60 between the side flows 67 and the carrier fluid 45 are shown as dashed lines in FIG. 16. The side flows are not intended to mix with the carrier fluid, although some mixing and turbulence may occur along these boundaries on a small scale without adversely affecting the performance of the device. Similar to the microchannel delivering the carrier fluid, the opposed flow channels are arranged to deliver the side flows in a laminar state. The fluid comprising both the carrier and the side flows may be a physiological buffer at physiological salt concentrations and pH that is suitable for most polymers, such as DNA or RNA. Both the side flows and the carrier fluid are usually similar fluids, although different fluids may be used, for example, to maintain a better boundary between the fluids as they enter and flow through the microchannel together.

The opposed flow control channels allow additional fluid to be added to the microchannel. The additional fluid can focus the carrier fluid in the microchannel and create a velocity gradient in the microchannel. The microchannel has a generally constant cross-sectional area along its length, from the first end 50 to the second end 52 although other configurations are possible. As fluid enters the microchannel from the opposed flow control channels, 54, 56, it reduces the cross-sectional/area available to the carrier fluid. Both the carrier fluid and the side flow fluids are generally incompressible. Therefore, to compensate for the additional fluid, the net velocity of the carrier fluid at the second end 52 may be greater than the net velocity of the fluid at the first end carrier 50 to maintain a balance between the volume of flow in and the flow out of the carrier fluid through the microchannel. The introduction of fluid from the opposed flow control channels effectively reduces the cross-sectional area available to the passing carrier fluid. This creates a focusing effect and a velocity gradient as discussed above, that can be used to manipulate a polymer in the carrier fluid. Both the carrier fluid and the fluid entry from the apposed flow control channels 54, 56 are generally characterized by parallel flowstreams once they pass the downstream edge of the apposed flow control channels. As shown in FIG. 15, the side flows create a fluidic funnel at the boundaries 58, 60 with the carrier fluid. This funnel reduces the cross-sectional area available to the carrier fluid. This, in turn, causes the streamlines of the carrier fluid to be focused and accelerated.

A polymer contained in the carrier fluid that enters this velocity gradient will be aligned or stretched and focused as discussed previously. A polymer entering the velocity gradient will be focused in a direction perpendicular to the flow and aligned or elongated in a direction parallel to the flow so that it can be directed accurately towards a location in the cross-section of the channel as desired. Such locations may include detection zones 62 as shown in FIG. 16. Detection zones may be used to perform actual analysis on the polymer or may simply be used to detect the presence of the polymer at a location in the microchannel. The detection zones are shown situated in the middle of the microchannel. However, they may alternately be situated at various points across the width of the microchannel or they may encompass the entire width of the microchannel. Other detection zones may be capable of being moved to a desired position or may also be capable of being actively focused to a desired size. Tradeoffs generally exist between the size and performance capabilities of most detection zones, that is, a smaller detection zone may be better adapted to detect or analyze a polymer that passes through it, but then a polymer is less likely to pass through a smaller detection zone. In order to detect or image a polymer as if it were in a quiescent pool, the detection zone may also be arranged to move at the same velocity as the passing fluid. This will allow the polymer to appear to the detection zone as if it were standing still.

The boundaries 58, 60 between the side flows and the carrier fluid generally define the shape of a funnel. This funnel begins where the side flows are introduced into the microchannel at the upstream edge of the opposed flow control channels. It continues reducing the cross-sectional area available to the carrier fluid in downstream positions until a minimum cross-sectional area for the carrier fluid is achieved. This minimum cross-sectional area is called the throat 69 of the funnel and is usually achieved at a point in-line with the downstream edge of the opposed flow control channels. Beyond the throat, the carrier fluid may generally form a uniform velocity laminar flow with the side flows. Again, there may exist some turbulent sections or mixing near the edges of the microchannel which generally do not adversely affect the performance of the device. The distance between the throat and the beginning of the funnel, in this case the upstream edge of the opposed flow channels, divided by the diameter or largest cross-sectional dimension of the funnel is known as the funnel aspect ratio. The ratio of the cross-sectional area of the microchannel over the cross-sectional area of the throat is known as the funnel reduction ratio. The funnel reduction ratio is a factor that can be adjusted by changing factors associated with each of the carrier fluid or the side flows such as the flow rates.

A polymer entering the velocity gradient in the microchannel will be manipulated by fluid in the gradient until it has passed through the velocity gradient and enters a downstream uniform velocity laminar flow zone. Therefore, if a detection zone is to image an entire, aligned or elongated polymer for analysis after it has been completely manipulated by a velocity gradient, the detection zone should be located downstream of the velocity gradient by at least a distance equal to one full length of the polymer. This is because the polymer will continue to be manipulated until the last portion has exited the velocity gradient, meaning that the forward most portion could be one full polymer length downstream. Also, the fluidic drag force that acts on the polymer while it is in the velocity gradient may have stretched the polymer elastically beyond its contour length (i.e., overstretched the polymer). This elastic stretching may recover when the polymer has exited the velocity gradient, depending on various factors, such as relaxation rate and flow rate to name a couple.

In some embodiments, the flow rate of the side flows may be modulated by a user to adjust the acceleration of the velocity gradient or the position of the velocity gradient in the microchannel. If the flow rate of the side flows is increased relative to the carrier fluid, the cross-sectional area available for the carrier fluid at downstream locations, including the throat, will be reduced. This reduced cross-sectional area will increase the flow velocity of the carrier fluid at these points. This will also reduce the funnel reduction ratio. Modulation of the side flows can occur while a polymer is being delivered through the microchannel to adjust to the specific polymer or it may occur prior to polymers being delivered down the microchannel. Similar effects may also be achieved by adjusting the flow rate (or another parameter) of the carrier fluid alone or in conjunction with the side flows. It is also possible to modulate the flow rate of one side flow relative to the other side flow. For instance, increasing the flow rate of the upper side flow relative to the lower side flow, all else constant, will move the throat of the velocity gradient toward the lower side wall of the microchannel. Moving the throat in this manner can be used to position a polymer contained therein in a desired lateral point of the microchannel. This again may be used to move the polymer into a detection zone or to move it in line with another device at a downstream position used to manipulate the polymer for subsequent analysis.

FIG. 15 shows one embodiment of the opposed flow control channels entering a microchannel, which is an example of an aspect of the invention and is not limiting. Other embodiments may accomplish the same task as the embodiment shown in FIG. 15 while being constructed in a different manner. For instance, the embodiment of FIG. 16 has the opposed flow channels angled with respect to the microchannel. Such a configuration may minimize the potential for turbulence at the intersection between the microchannel and the opposed flow control channels. The microchannel of FIGS. 15 and 16 are shown to have a constant cross-sectional area throughout their length, however, other embodiments may gradually increase or decrease the cross-sectional area of the microchannel to accomplish different effects. For instance, a microchannel that reduces its cross-sectional area at points downstream will serve to create a velocity gradient itself thereby amplifying the acceleration of any gradient created by the opposed side flow channels. A microchannel with cross-sectional area that increases at points downstream will serve to attenuate the severity of the velocity gradient created by the opposed flow control channels.

The opposed flow channels are described as being opposed; however, it is not a requirement that they be directly opposed to one another. Embodiments can exist where the opposed flow control channels are staggered at different positions of the microchannel side walls. Such an arrangement may cause the carrier fluid to bend about each entering side flow before creating the velocity gradients like those shown in FIG. 15. This bending of the carrier fluid may be used to push the polymer contained therein towards one side or another of the carrier fluid. Still in other embodiments, only one opposed channel may be used or opposed channels of different configurations may be used. In such embodiments, the velocity gradient may exist skewed to one side of the microchannel. In the case of only one opposed channel, the funnel will appear as one boundary reducing the cross-sectional area between the carrier fluid and the opposite side wall as the carrier fluid progresses downstream. In this embodiment, increasing the flow rate of the side flow will serve to focus the carrier fluid more closely towards the side wall in addition to increasing acceleration of the velocity gradient.

A different type of microfluidic device is shown in FIG. 17. This device again comprises a microchannel, typically embedded in a silicon chip and covered with a cover slip. There is a primary microchannel with a first end 50 and a second end 52 and an elongation zone 70 disposed there-between. Two opposed polymer control channels 64 and 66 intersect the side walls 65 of the microchannel. As in the previous device, a carrier fluid capable of containing a polymer or polymers is delivered from the first end 50 of the microchannel toward the second end 52 with the carrier fluid in a laminar state. A second opposed fluid is delivered from the second end 52 of the microchannel toward the first end 50. The second fluid is also in a primarily laminar state. These two flows can interact between the opposed polymer control channels where both the carrier fluid and the opposed flow each separate into two different flows, each following one of the opposed polymer control channels 64, 66 away from the microchannel. This interaction can create a stagnation point 68 centered generally between the intersection of the microchannel and the opposed polymer control channels. As previously discussed, the stagnation point 68 is a point in the fluid characterized by low or no flow velocities. A fluid approaching the stagnation zone can remain in a laminar state and separate in an elongation zone 70 upstream from the stagnation point, subsequently flowing into one of the two opposed polymer control channels. A polymer contained in the streamlines of the carrier fluid that are separated by the elongation zone will continue to follow the separating streamlines as they proceed down respective opposed polymer control panels. As the streamlines separate further, they can elongate the polymer in a direction parallel with the separated streamlines.

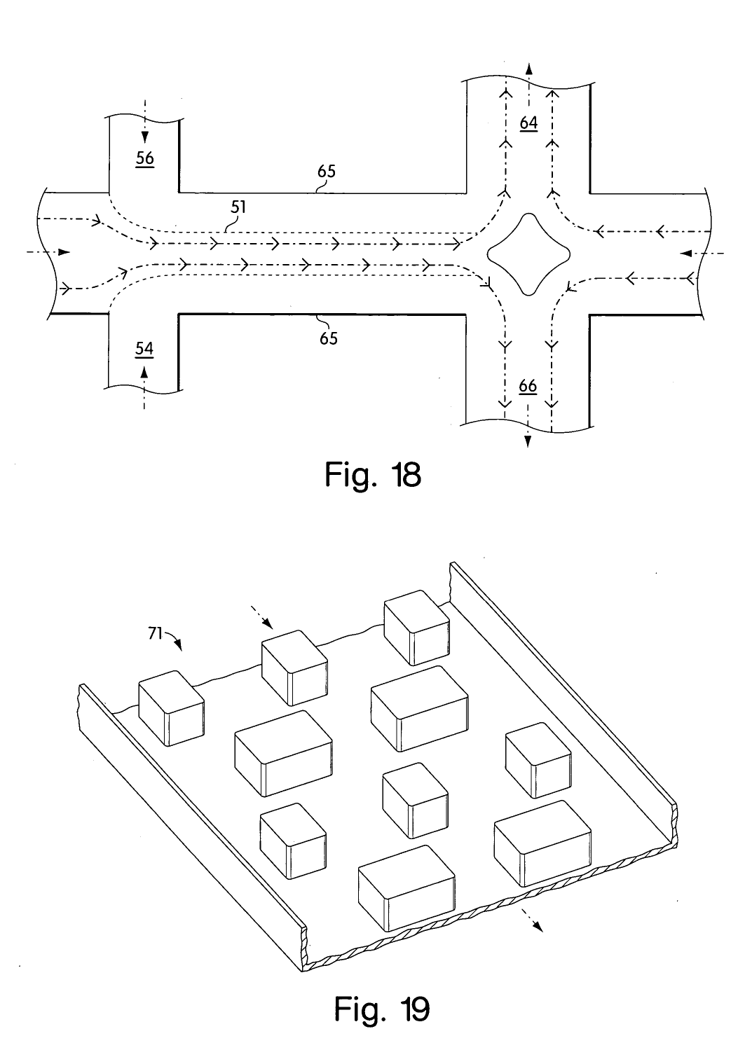

A polymer may be aligned and/or elongated in the flow moving away from the stagnation point, if a polymer is aligned with substantially equal portions of polymer on either side of the stagnation point as it approaches the stagnation point. A focusing device as the previously discussed may be used to position a polymer such that it will have substantially equal portions on either side of the stagnation point. Such a focusing device in combination with opposed polymer control channels is shown in FIG. 18. The elongation zone 70 associated with the stagnation point is a useful tool for elongating a polymer because it is less sensitive than other microfluidic phenomenon to the initial arrangement of the polymer entering it. For instance, the elongation zone can elongate and align a polymer whether it is introduced to an elongation zone in a coiled state, hairpin state, or a somewhat aligned state.

If a polymer approaches the stagnation point with a majority of the polymer situated to one side of the stagnation point (for instance, with the majority nearer to polymer control channel 64), the majority will likely be pulled by the fluid of a first polymer control channel 64 while the remaining portions of the polymer will be pulled by the fluid flowing into a second polymer control channel 66. As the portions of the polymer progress down each of these polymer control channels, the net fluidic drag force acting on the majority of the polymer will likely overcome the much lower net fluidic drag force acting on the remaining portions of polymer in the second polymer control channel 66. The net fluidic drag force from fluid flowing into the second polymer control channel 66 may be enough to pull the entire polymer into an aligned, elongated state. However, in other situations, it may not be enough to accomplish this and a portion of the polymer in the first polymer control channel 64 may remain in a unaligned state as the entire polymer travels away from the stagnation point 68 in the first polymer control channel 64.

The elongation zone 70 can be adapted to accommodate polymers that enter it with less than substantially equal portions of the polymer arranged on either side of the stagnation point. This is usually accomplished by adjusting the relative flow rates of fluid in the polymer control channels 64, 66. The flow rate of the first polymer control channel can be reduced with respect to the second polymer control channel 64 when the polymer is located within the polymer control channels 64, 66. This will reduce the net fluidic drag force acting on the portion of the polymer in the first polymer control channel 64 thereby allowing the net fluidic drag force associated with the second polymer control channel 66 to bring the polymer to an aligned or elongated state. This same action of decreasing the flow rate of the first polymer control channel can also move the stagnation point 68 closer to the first polymer control channel. Adjusting the flow rates of the polymer control channels in this manner can move the stagnation point closer to either of the polymer control channels so that a polymer approaches it with substantially equal portions on either side of the stagnation point. While these examples involved decreasing the flow of the first polymer control channel, the flow in the second control channel could also be increased to achieve similar results with respect to the second polymer control channel.

Adjusting the flow rates of the polymer control channels with respect to one another can also be used to hold an elongated polymer so that it may be analyzed. In this scenario, the net fluidic drag force associated with portions of the polymer in both the first and second polymer control channels are set substantially equal to one another by adjusting their respective flow rates. Setting these forces equal to one another serves to prevent the polymer from being moved with respect to the microchannel. However, since fluid is still moving with respect to the polymer, the fluidic drag forces can still align or elongate the polymer. These methods can be used to hold a polymer near the stagnation point in an aligned or elongated state for analysis or simply to align the polymer such that it can be delivered downstream for subsequent manipulation or analysis in an aligned state or elongated. Similar to what occurs with a velocity gradient, an elongated polymer that completely exits the elongation zone and then enters a uniform velocity laminar flow field may relax to an aligned/partially stretched or may remain in an elongated/stretched state but will generally not remain in an elongated/over stretched state.

Other embodiments of the invention accomplish a similar elongating effect by simply placing obstacles within the carrier fluid. For instance, FIG. 5 shows what could be a cylindrical obstacle 34 extending from the floor of a microchannel. The approaching fluid creates an elongation zone and a stagnation point at a central point of the object facing the flow. The streamlines then separate and travel around the cylindrical obstacle 34 coming back towards one another at an opposite side if the flow remains laminar. The streamlines may not come back together if the flow turns turbulent. Whether or not the polymer is caught on this stagnation point and uncoiled or stretched is dependent upon its placement in the flow when it approaches the object 34. If substantially equal portions of the polymer approach the stagnation point and of the obstacle 34 on opposite sides, then the polymer will likely find portions extending downstream on either side of the object34. As polymer approaches the obstacle, it will enter the stagnation point and likely pass through until it makes contact with the obstacle 34 itself. The portions on either side of the obstacle will continue downstream following streamlines until they are not permitted to do so by the contact between the polymer and the obstacle. At this point the streamlines in which they reside will apply fluidic drag forces against the portions of the polymer, thereby aligning or elongating them and placing the polymer in a hairpinned state.

It is possible that a polymer could contact an object with substantially equal portions of the polymer on either side of the object 34. The portions may subsequently experience substantially equal net fluidic drag forces; therefore holding the polymer against the obstacle in an elongated state. However, it is more likely that one of the net fluidic drag forces associated with a portion of the polymer on one side of the obstacle will be at least slightly greater than the net fluidic drag force associated with the opposite portion of the polymer on the other side of the obstacle. In this case, the greater of the net drag forces will pull the entire polymer around the obstacle until it is free of the obstacle and can continue downstream on the side associated with the greater net fluidic drag force. In this sense, the obstacle “transiently tethers” the polymer for a period of time.

Transient tethering is useful for several reasons. First, it can serve to arrange a coiled polymer into an aligned or partially aligned state so that it can be delivered downstream in this state for analysis or subsequent manipulation. Second, it temporarily holds a polymer such that it can be analyzed. The obstacle described above is cylindrical; however, this cylindrical obstacle is intended to be exemplary and not limiting. Any of a large variety of shapes could equivalently serve a similar purpose. Additionally, other shapes may provide for easier manufacturability. Some alternative shapes may include square cross-sections, rectangular cross-sections as shown in FIG. 19, elliptical cross-sections, and V-shaped cross-sections such as discussed in U.S. Pat. No. 5,837,115 which is hereby incorporated by reference. These obstacles may be placed within a microchannel, upstream of opposed flow control channels as previously discussed. In this manner, they can serve to pre-orient the polymer so that it enters the velocity gradient in at least a semi-aligned state. Multiple obstacles may be placed across the channel or in a matrix like fashion to create an obstacle field 71 as shown in FIG. 19. In other embodiments, multiple obstacles may be arranged in irregular patterns within the microchannel as the invention is not limited in this respect. Such an obstacle field increases the probability that a polymer will interact with one of the obstacles. The obstacle fields may comprise rows that are staggered with respect to one another, they may be spaced consistently or differently, they may contain different sized or shaped obstacles, as the invention is not limited in this respect either. Additionally, a detection zone may be placed adjacent to any of the obstacles in the obstacle field, or it may encompass the entire obstacle field.

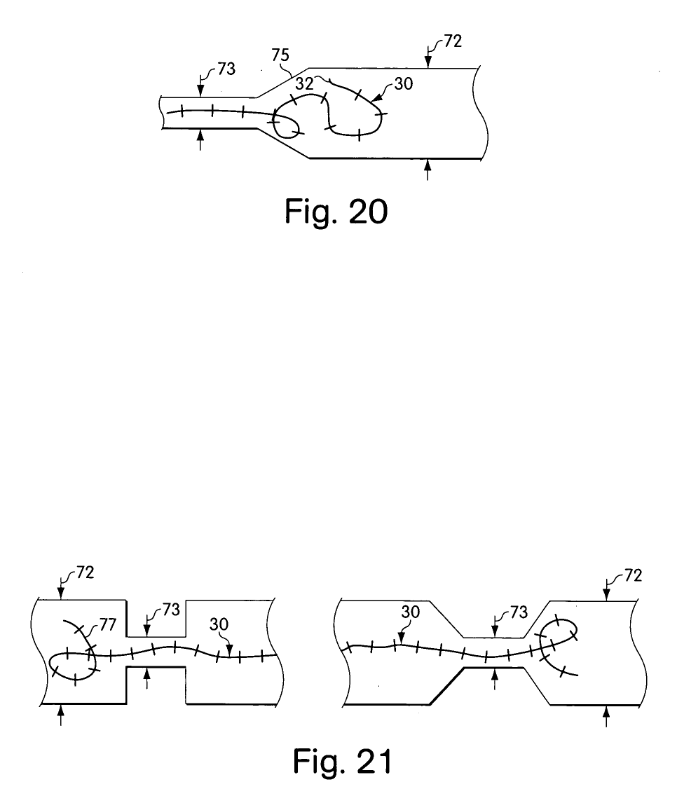

Once a polymer has been placed in an aligned or elongated state, it may be desirable to hold it in that state for prolonged analysis, multiple analysis steps and/or subsequent polymer manipulation. While this can be accomplished by the opposed polymer control channels as discussed above, it is desirable in some scenarios to hold the polymer in the aligned state where low flow, or no-flow fluid surroundings can exist. A device that accomplishes this effect is shown in FIG. 20. This device includes a microchannel having opposed side walls defining a first microchannel dimension 72 and opposed side walls defining a second narrower microchannel dimension 73. The transition 75 between these two dimensions is shown as a straight slant; however, this could also comprise a wall perpendicular to each section, a smoothly curved surface, or any other configuration as the invention is not limited in this respect.

One method of retaining a polymer in an aligned and/or elongated state is now described. The carrier fluid delivers a polymer in between the walls defining the narrower dimension 73 in a substantially aligned or elongated state. A first end 32 of the polymer extends through the substantially narrower dimension 73 in the microchannel and into the first microchannel dimension 72. The flow then slows or stops leaving the polymer substantially still relative to the microchannel. The first end 32 it is allowed to return to a higher entropy, coiled state in a natural manner when it extends into the portion of the microchannel defined by the first dimension 72. This usually includes the polymer first coiling at its end to form a shape reminiscent of a barbell. After a period of time, the first end 32 becomes a coiled end 77 of the polymer which will prevent it from traversing back through the narrower portion 73 of the microchannel. This will occur as long as the forces pulling the polymer back through the narrower portion are not great enough to uncoil the polymer. When an attempt is made to pull the polymer back through the narrower portion 73 of the channel, the coiled end 73 of the polymer will contact the transition 75 between the narrow width 73 and the larger width 72. This contact will create a force that resists the polymer being pulled through the narrower channel dimension. This combination of a narrow and substantially larger channel width is referred to herein as a crimp. Such a crimp may be used alone at a point in a microchannel to retain an end of a polymer or two may be used to hold opposed ends of a polymer in an aligned and/or elongated state. Usually, a polymer such as DNA or RNA is held in a stretched state, although they can also be held in a partially stretched or over stretched state. In some embodiments several crimps may be used in multiple places throughout a microchannel to enable the channel to hold different portions of a polymers, or polymers of varying length.

FIG. 21 shows two cutaway views of an arrangement with two crimps holding the opposite ends of a polymer 30 disposed therein. This device can be used effectively to hold a polymer delivered through the microchannel in an aligned and/or elongated state once each of its ends are placed within a crimp. When a polymer is held with each of its ends disposed in a crimp, the ends of the polymer will naturally begin to coil. These coiled ends will contact the transition wall 75 of the crimp where the contact will prevent the polymer from coiling further or traversing back through the crimp. While the polymer is held in the aligned and/or elongated state, it may be analyzed, or other processing steps may be performed on the polymer such as dialysis or the attachment of additional probes. Numerous crimps may be placed throughout the length of the microchannel to increase the range of polymer lengths that a microchannel with crimps can hold.

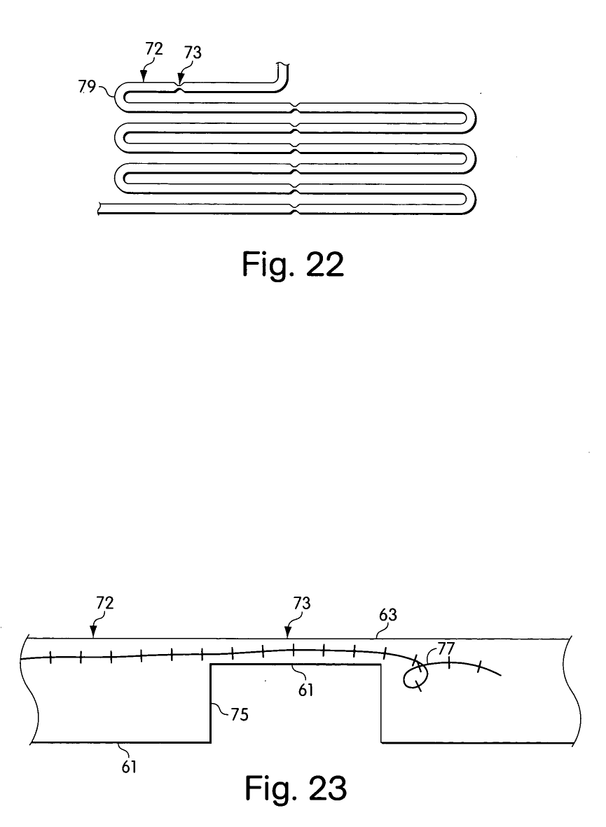

Additional arrangements of microchannels with crimps may exist in serpentine fashion as shown in FIG. 22. Serpentine arrangement of the microchannel may serve to limit the amount of space required to hold polymers of great length on a single microchip in some embodiments. In some cases, the corners 79 may further inhibit the relaxation of the polymer. While FIGS. 20, 21 and 22 show crimps as being differences in the widths of various side channels, the invention is not limited in this respect. For instance, FIG. 23 shows a crimp that exists between the cover 63 and the bottom wall 61 of a microchannel. In order to remove the polymer from the crimps, the fluid need only be returned to a flow rate that can apply a large enough fluidic drag force against the polymer to align the polymer and pull it through the crimp or to break it and free it from the crimps.

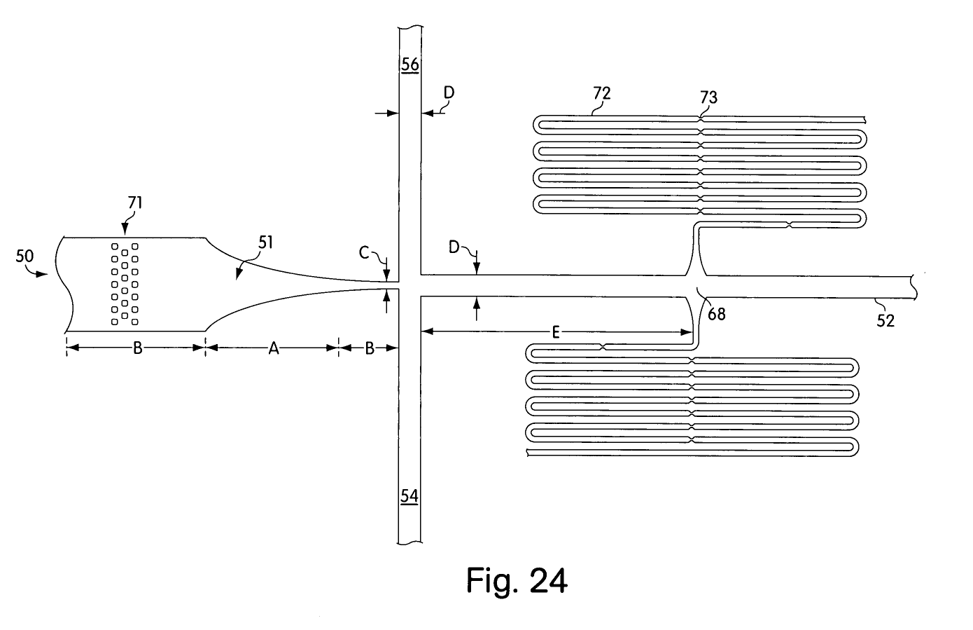

The various microfluid devices of this invention are discussed independently as they may be employed independently in any microfluidic device. However, they may also be combined in any fashion into a single microfluidic device. For instance, FIG. 24 shows a single microfluidic device that comprises a plurality of obstacles near the first end 50 of the channel, opposed flow control channels 54, 56 in a portion of the microchannel downstream from the obstacles for focusing flow and creating a velocity gradient, flow emanating from a second end 52 of the microchannel for impinging on the flow from the first end of the microchannel to create a stagnation point 68 and associated elongation zone 70, opposed polymer control channels 64, 66 for manipulating the elongation zone 70 or a polymer, and downstream from each of the opposed polymer control channels serpentine portions exists with crimps disposed therein for retaining a polymer. Detection zones may be placed at any points within this entire microfluid device to detect or image or analyze the polymer located in the detection zone.

The various microfluidic devices may be implemented in microchannels or other devices of many different dimensions. However, the various features represented in represented in FIG. 24 may have be implemented in one particular embodiment with dimensions ‘A’ through ‘E’ as represented below. However, other dimensions may be used as the invention is not limited in this respect.

| Dimension | Size (microns) |
|---|---|
| A | 90 |
| B | 100 |
| C | 1 |
| D | 10 |
| E | 128 |

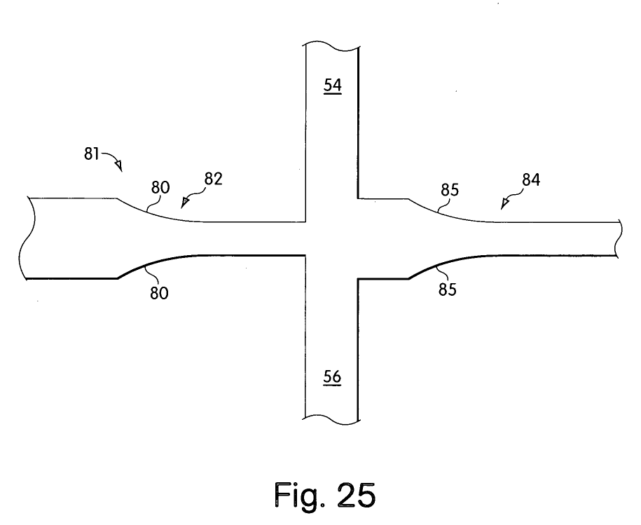

FIG. 25 shows another example of how various microfluidic devices of the present invention may be combined in a particular fashion. FIG. 25 shows a first section 81 of a microchannel that has substantially opposed sidewalls 80 forming a funnel shape 82. A carrier fluid passing through this section of the microchannel will be focused, such that any polymer contained therein will be elongated and/or aligned. At a position downstream of the first section, two opposed flow controlled channels 54, 56 intersect with the microchannel. Each of these opposed flow control channels 54, 56 inject a side-flow that can be used to move the carrier fluid to a desired position within the channel cross-section. For instance, the side-flows may be used to locate the polymer such that it passes through the center of a downstream detection zone. Also, the side-flows may be used to create a second velocity gradient for focusing (meaning either aligning or elongating) the polymer in the carrier fluid passing therethrough. Still, at a position downstream of the opposed flow control channels, another section 84 exists with substantially funnel shaped opposed walls. These opposed walls 85, like those of the first section, create another velocity gradient for further focusing the carrier fluid and polymer contained therein as they pass through this section. At a position downstream of this third section, or anywhere else within the microchannel, a detection zone may be located to perform any of the above previously described analysis on the polymer.

Generally each end of the microchannel or of the channels intersecting with the primary microchannel may terminate in an opening that extends outside of the microchip and into a microchip manifold. These openings may be in fluid communication with a mating opening in the apparatus designed to contain the reusable chip holder and chip which are optionally re-useable. Flow through each of these apertures and ultimately in the respective microchannels may be controlled by any flow control devices known to those in the art. Such devices may include vacuum pumps, positive displacement pumps, pressure controlling pumps, or throttling valves used in conjunction with any of the previously mentioned devices. These devices may in turn be controlled directly by a user, or by a pre-programmed controller as the invention is not limited in this respect. The holder may control the position of the microchip such that when placed in the apparatus, the chip is located beneath an imaging device.

The methods of the invention can be used to generate unit specific information about a polymer by capturing polymer dependent impulses from the polymer using the microfluidic devices to manipulate the polymer. As used herein the term “unit specific information” refers to any structural information about one, some, or all of the units of the polymer. The structural information obtained by analyzing a polymer may include the identification of characteristic properties of the polymer which (in turn) allows, for example, for the identification of the presence of a polymer in a sample or a determination of the relatedness of polymers, identification of the size of the polymer, identification of the proximity or distance between two or more individual units or unit specific markers a polymer, identification of the order of two or more individual units or unit specific markers within a polymer, and/or identification of the general composition of the units or unit specific markers of the polymer. Since the structure and function of biological molecules are interdependent, the structural information can reveal important information about the function of the polymer.

A “polymer dependent impulse” as used herein is a detectable physical quantity which transmits or conveys information about the structural characteristics of a unit specific marker of a polymer. The physical quantity may be in any form which is capable of being detected. For instance the physical quantity may be electromagnetic radiation, chemical conductance, electrical conductance, etc. The polymer dependent impulse may arise from energy transfer, quenching, changes in conductance, radioactivity, mechanical changes, resistance changes, or any other physical changes.

The method used for detecting the polymer dependent impulse depends on the type of physical quantity generated. For instance if the physical quantity is electromagnetic radiation then the polymer dependent impulse is optically detected. An “optically detectable” polymer dependent impulse as used herein is a light based signal in the form of electromagnetic radiation which can be detected by light detecting imaging systems. In some embodiments the intensity of this signal is measured. When the physical quantity is chemical conductance then the polymer dependent impulse is chemically detected. A “chemically detected” polymer dependent impulse is a signal in the form of a change in chemical concentration or charge such as ion conductance which can be detected by standard means for measuring chemical conductance. If the physical quantity is an electrical signal then the polymer dependent impulse is in the form of a change in resistance or capacitance. These types of signals and detection mechanisms are described in U.S. Pat. No. 6,355,420 B1.

The polymer dependent impulses may provide any type of structural information about the polymer. For instance these signals may provide the entire or portions of the entire sequence of the polymer, the order of polymer dependent impulses, or the time of separation between polymer dependent impulses as an indication of the distance between the units or unit specific markers.

Having described several embodiments of the invention in detail, various modifications and improvements will readily occur to those skilled in the art. For instance, any of the microfluidic devices of the present invention may be used in combination with any other devices, such as the electrical devices described herein, or any know devices or methods. Such modifications and improvements are intended to be within the spirit and scope of the invention. Accordingly, the foregoing description is by way of example only and is not intended as limiting. The invention is limited only as defined by the following claims and the equivalence thereto.

1. An apparatus for positioning a polymer in a microchannel, which comprises:
a microchannel having first and second ends and substantially opposed sidewalls, the microchannel being constructed and arranged to transport a polymer carrier fluid such that, when present, the polymer flows from the first end toward the second end in a laminar flow stream;
a first section of the microchannel disposed between the first and second ends of the microchannel, the substantially opposed sidewalls of the first section constructed and arranged to create a first velocity gradient in the flow stream passing there through;
opposed flow control channels in fluid communication with the microchannel, the flow channels being positioned between the first section and the second end of the microchannel;
a flow controller to control the flow of fluid through the opposed flow control channels to maintain the flow stream containing the polymer in a laminar state isolated from the substantially opposed sidewalls of the microchannel at points downstream from the opposed flow control channels;
a second section of the microchannel disposed between the opposed flow control channels and the second end of the microchannel, the substantially opposed sidewalls of the second section being constructed and arranged to create a second velocity gradient in the flow stream passing there through; and
a detection zone disposed within the microchannel.

2. The apparatus of claim 1 wherein the flow controller is adapted to move the polymer into the detection zone.

3. The apparatus of claim 1 wherein the flow controller comprises at least two flow controllers, each of the at least two controllers for independently controlling the flow of fluid through each of the opposed flow control channels.

4. The apparatus of claim 1 wherein the flow controller comprises a pressure source.

5. The apparatus of claim 1 wherein the substantially opposed sidewalls of the first section are substantially non-parallel.

6. The apparatus of claim 5 wherein the substantially opposed sidewalls of the second section are substantially non-parallel.

7. The apparatus of claim 1 wherein the second velocity gradient ends upstream of the detection zone by at least a distance equal to the polymer.

8. The apparatus of claim 6 wherein the polymer is DNA.

9. The apparatus of claim 7 wherein the polymer is RNA.

10. The apparatus of claim 7 adapted to create a fluidic boundary between the carrier fluid an the flow through the opposed flow control channels wherein the opposed flow controller is further adapted to control a shape of the fluidic boundary.

11. A method of positioning a polymer within a microchannel, the method comprising:
providing a polymer positioning apparatus comprising:
a microchannel having first and second ends and substantially opposed sidewalls, the microchannel being constructed and arranged to transport a polymer carrier fluid such that, when present, the polymer flows from the first end toward the second end in a laminar flow stream;
a first section of the microchannel disposed between the first and second ends of the microchannel, the substantially opposed sidewalls of the first section constructed and arranged to create a first velocity gradient in the flow stream passing there through;
opposed flow control channels in fluid communication with the microchannel, the flow channels being positioned between the first section and the second end of the microchannel;
a flow controller to control the flow of fluid through the opposed flow control channels to maintain the flow stream containing the polymer in a laminar state isolated from the substantially opposed sidewalls of the microchannel at points downstream from the opposed flow control channels; and
a second section of the microchannel disposed between the opposed flow control channels and the second end of the microchannel, the substantially opposed sidewalls of the second section being constructed and arranged to create a second velocity gradient in the flow stream passing there through;
providing a polymer carrier fluid containing a polymer into the microchannel; and
manipulating the flow controller for selectively positioning the polymer within the microchannel.

12-16. (canceled)

17. A method of focusing a polymer within a microchannel, the method comprising:
providing a carrier fluid containing a polymer to a microchannel adapted to deliver the carrier fluid from a first end of the microchannel to a second end of the microchannel;
focusing the carrier fluid in a first velocity gradient created by a first set of substantially opposed walls of the microchannel; then
focusing the carrier fluid in a second velocity gradient created by a side flow of fluid entering the microchannel; and then
focusing the carrier fluid in a third velocity gradient created by a second set of substantially opposed walls of the microchannel.

18-22. (canceled)

23. An apparatus for elongating a polymer which comprises:
a microchannel having first and second end, a polymer elongation zone, and opposed sidewalls, the microchannel being constructed and arranged to transport a polymer carrier fluid such that, when present, the polymer flows from the first end toward the polymer elongation zone in a laminar flow stream;
opposed flow control channels in fluid communication with the microchannel through the opposed sidewalls, the flow control channels being positioned between the first end of the microchannel and the polymer elongation zone;
opposed polymer control channels in fluid communication with the microchannel through the opposed sidewalls, the polymer control channels defining the polymer elongation zone and being positioned between the opposed flow control channels and the second end of the microchannel;
a first end fluid controller for directing a fluid through the microchannel from the first end toward the polymer elongation zone;
an opposed flow controller for controlling the flow of fluid through the opposed flow control channels to maintain the flow stream containing the polymer in a laminar state isolated from the opposed sidewalls of the microchannel;
an opposed polymer channel controller for controlling the flow of fluid through the opposed polymer control channels, and
a second end flow controller for directing fluid through the microchannel from the second end toward the polymer elongation zone.

24-27. (canceled)

28. A method for elongating a polymer which comprises:
providing a polymer elongation apparatus comprising:
a microchannel having a first end, a polymer elongation zone, and opposed sidewalls, the microchannel being constructed and arranged to transport polymer carrier fluid such that, when present, the polymer flows from the first end toward the polymer elongation zone in a laminar flow stream;
opposed flow control channels in fluid communication with the microchannel through the opposed sidewalls, the flow control channels being positioned between the first end of the microchannel and the polymer elongation zone;
opposed polymer control channels in fluid communication with the microchannel through the opposed sidewalls, the polymer control channels defining the polymer elongation zone and being positioned between the opposed flow control channels and the second end of the microchannel;
an opposed flow controller for controlling the flow of fluid through the opposed flow control channels to maintain the flow stream containing the polymer in a laminar state isolated from the opposed sidewalls of the microchannel; and
an opposed polymer channel controller for controlling the flow of fluid through the opposed polymer control channels
directing a fluid carrier containing the polymer to be elongated through the microchannel from the first end toward the polymer elongation zone in a laminar flow stream; and
directing a flow control fluid through the opposed flow control channels into the microchannel in a manner such that polymer-containing flow stream is isolated from the sidewalls of the microchannel.

29. (canceled)

30. An apparatus for maintaining a polymer in an elongated configuration which comprises:
a microchannel constructed and arranged to contain a polymer in a carrier fluid, the microchannel having opposed sidewalls defining a first microchannel width, a second microchannel width, smaller than the first width, and a transition between the first and second microchannel widths;
wherein the transition adapted to contact and inhibit relaxation of an elongated polymer contained within the first microchannel width.

31. An apparatus for elongating a polymer and maintaining it in an aligned or elongated configuration the apparatus comprising:
a microchannel having first and second ends, a polymer elongation zone, and opposing sidewalls, the microchannel being constructed and arranged to transport a polymer in a carrier fluid such that, when present, the polymer flows from the first end toward the polymer elongation zone in a laminar flow stream;
opposed polymer control channels in fluid communication with the microchannel through the opposing sidewalls, the polymer control channels adapted to provide a flow of fluid for defining the polymer elongation zone, the polymer control channels positioned between the first end and the second end of the microchannel, wherein at least one of the polymer control channels includes at least one transition to a narrower microchannel width, the transition for contacting and inhibiting relaxation of an elongated or aligned polymer contained in the narrower width, and further wherein at least one of the polymer control channels includes at least one serpentine bend to cause at least one portion of the polymer control channel to be located adjacent and parallel to another portion of the polymer control channel;
a first end fluid controller for directing a fluid through the microchannel from the first end toward the polymer elongation zone;
an opposed polymer channel controller for controlling the flow of fluid through the opposed polymer control channels; and
a second end fluid controller for directing fluid through the microchannel from the second end toward the polymer elongation zone.

32-35. (canceled)

36. An apparatus for detecting a polymer comprising:
a microchannel having first and second ends;
an obstacle field arranged between the first and second ends at the microchannel, the microchannel being constructed and arranged to transport the polymer in a carrier fluid such that, when present, the polymer flows from the first end, through the obstacle field and toward the second end in a laminar flow; and
a detection zone located in the obstacle field, the detection zone for detecting the polymer.

37-42. (canceled)

43. A method for detecting a polymer which comprises:
providing an apparatus comprising a microchannel having first and second ends and an obstacle field between the first and second ends, the microchannel being constructed and arranged to transport the polymer in a carrier fluid such that, when present, the polymer flows from the first end, through the obstacle field and toward the second end in a laminar flow;
providing a polymer carrier fluid containing a polymer to be detected;
flowing the polymer in the carrier fluid through the obstacle field in a manner such that at least one polymer becomes transiently tethered to at least one obstacle comprising the obstacle field; and
detecting the transiently tethered polymer.

44-50. (canceled)

51. An apparatus for holding a polymer on a microchip, the apparatus comprising:
a microchannel disposed on the microchip, the microchannel having a first end and a second end and opposing sidewalls, the microchannel being constructed and arranged to transport a polymer in a carrier fluid, such that, when present, the polymer flows from the first end toward the second end along a flow path;
the microchannel being arranged on the microchip with a first bend causing a first portion of the microchannel to be located adjacent to and aligned with a second portion of the microchannel.

52-55. (canceled)
